(19) United States
(12) Plant Patent Application Publication
(10) Pub. No.: US 2016/0219764 P1
(43) Pub. Date: Jul. 28, 2016

(54) Rose AAC 362

(50) ;

(71) Applicant: Her Majesty the Queen in Right of Canada, as represented by the Minister of Agriculture & Agri-, Ottawa (CA)

(72) Inventor: Larry Dyck, Morden (CA)

(21) Appl. No.: 14/756,070

(22) Filed: Jul. 29, 2015

(30) Foreign Application Priority Data
Jan. 27, 2015 (CA) 15-8513

(51) Int. Cl. A01H 5/00

(52) U.S. Cl. PLT/134

(57) ABSTRACT

The invention is a new and distinct Rose variety denominated ‘AAC 362’. The new Rose is characterized by young leaves with purple coloration, and flowers having a bicolor bloom with petals that are red-pink with a light creamy-yellow center at opening that fades to white.

### LATIN NAME OF THE GENUS AND SPECIES OF THE PLANT CLAIMED

Rosa L.

### VARIETY DENOMINATION ‘AAC 362’

### BACKGROUND OF THE INVENTION

The new and distinct Rose variety described and claimed herein originated from a cross between a proprietary seedling ‘PO4’ (Meidiland×‘Frontenac’) and the ‘Yellow Submarine’ variety.

The female (seed) parent ‘PO4’ is a proprietary seedling derived from Meidiland x ‘Frontenac’.

The male (pollen) parent cv. ‘Yellow Submarine’ is a patented variety in the United States (U.S. Plant Pat. No. 16,659) derived from the ‘MACivy’ variety (U.S. Plant Pat. No. 8,362) which is sometimes known as the ‘Singin’ in the Rain’ variety, and an unreleased shrub rose designated R93412 from the breeding program of the patentee on U.S. Plant Pat. No. 16,659.

The present variety of Rose was first produced by controlled hybridization. The original cross was done at Agriculture and Agri-Food Canada, Morden Research Station, Manitoba, Canada in 2007. The hybrids from this initial cross were planted in field at the Morden Research Station in 2008.

‘AAC 362’ was first successfully asexually propagated by vegetative cuttings at the Morden Research Station in 2009 and the cuttings maintained in a greenhouse at the Morden Research Station. In 2010 and 2011, the cuttings were moved to the evaluation farms of Prairie Shade Nursery in Portage La Prairie, Manitoba, Canada.

The Rose variety ‘AAC 362’ is currently maintained in vitro by tissue culture at Phytoclone Inc., 1943 Rue Principale, St-Etienne-Des-Gres, Quebec. Stock plants for propagation are maintained by Adamson's Heritage Nursery Ltd, Langley, B. C., JC Bakker & Sons Wholesale Nurseries, St. Catharines, Ontario, Canada, and Jeffries Nurseries Ltd., Portage La Prairie, Manitoba, Canada. Material for long term preservation is maintained in the Rose germ plasm repository at Vineland Research and Innovation Centre, Vineland Station, Ontario, Canada.

### SUMMARY OF THE INVENTION

The new Rose variety ‘AAC 362’ differs from the reference varieties ‘Nicolas’ and ‘Champlain’ in the following characteristics:

Plants of the variety ‘AAC 362’ have new leaf growth that is purple, while plants of the reference variety ‘Nicolas’ have green leaves and plants of the reference variety ‘Champlain’ have deep green leaves.

Flowers of the variety ‘AAC 362’ have a bicolor bloom with petals that are red-pink with a light creamy-yellow center at opening that fades to white, compared to flowers of the reference varieties ‘Nicolas’ and ‘Champlain’ which have solid red blooms.

The characteristics most useful in distinguishing ‘AAC 362’ from the reference varieties are young leaf: purple coloration, and flower: bicolor bloom with petals that are red-pink with a light creamy-yellow center at opening that fades to white.

### BRIEF DESCRIPTION OF THE DRAWING

The accompanying photographic illustration illustrates in full color ‘AAC 362’. The colors are as nearly true as reasonably possible in color representation of this type. Colors in the photographs may differ from the color values cited in the detailed botanical description below, which accurately describes the colors of the new Rose.

FIG. 1 shows growth type and habit of ‘AAC 362’.

FIG. 2 shows young and mature stem of ‘AAC 362’.

FIG. 3 shows a comparison of young and mature prickles of ‘AAC 362’.

FIG. 4 shows a comparison of young and mature leaf of ‘AAC 362’.

FIG. 5 shows structure and color of the whole flower of ‘AAC 362’.

FIG. 6 shows fruit/hips of ‘AAC 362’.

FIG. 7 shows color of sepals and receptacle of ‘MC 362’.

FIG. 8 shows color of bud stage of ‘AAC 362’.

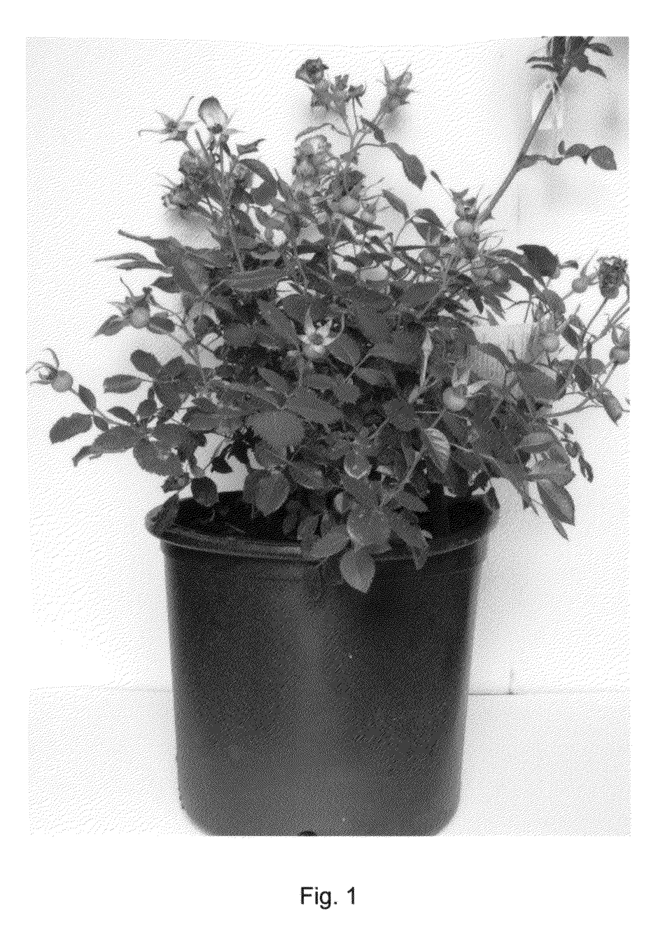

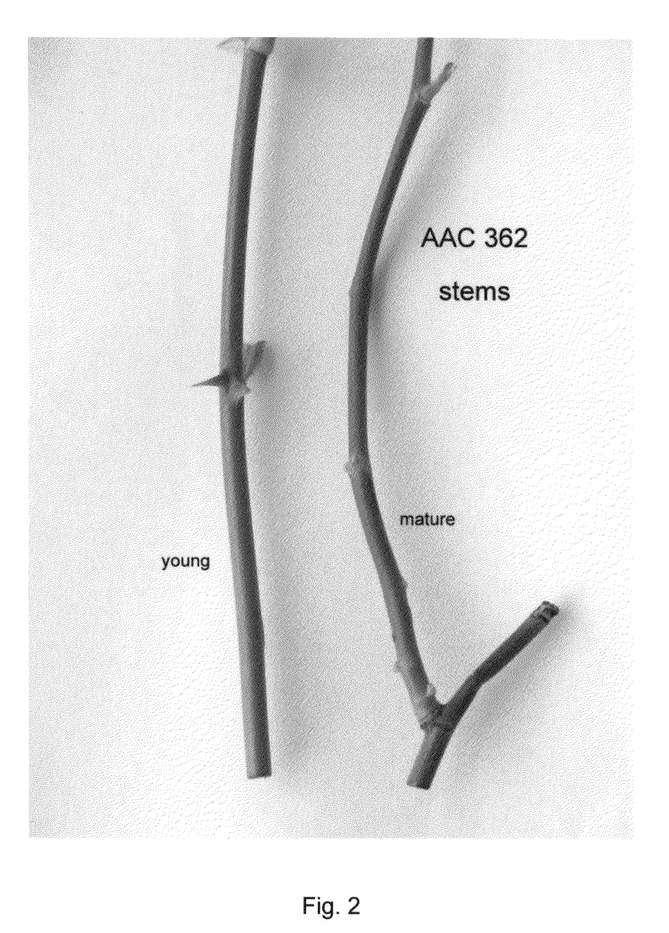

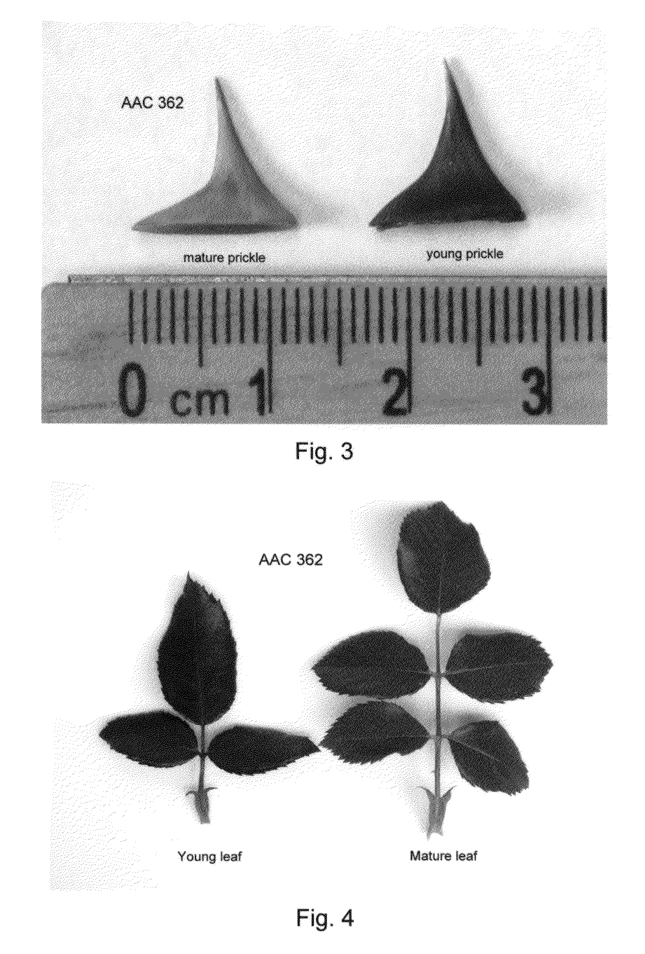

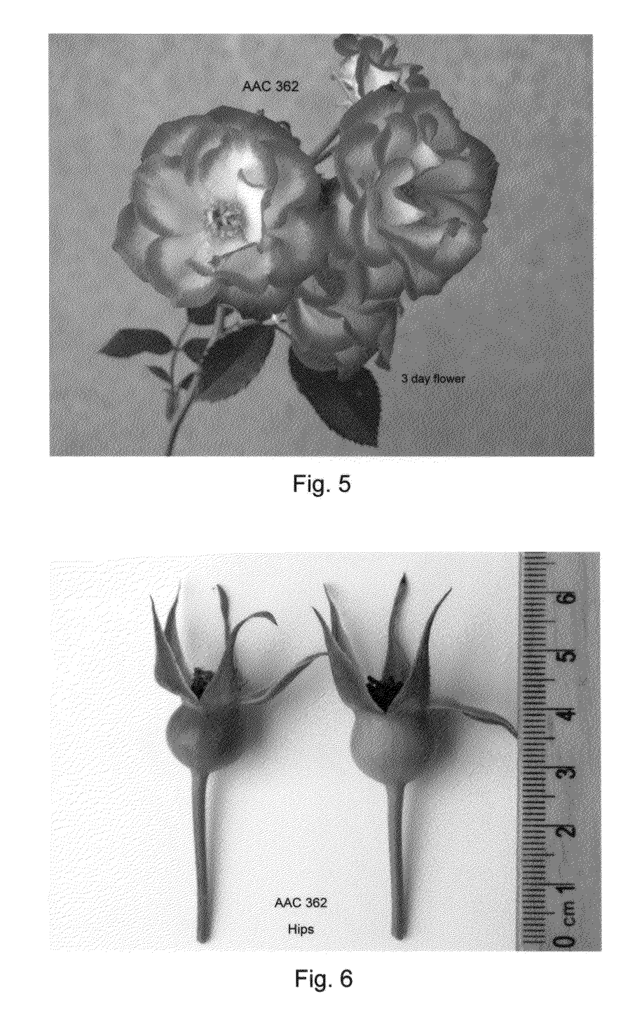

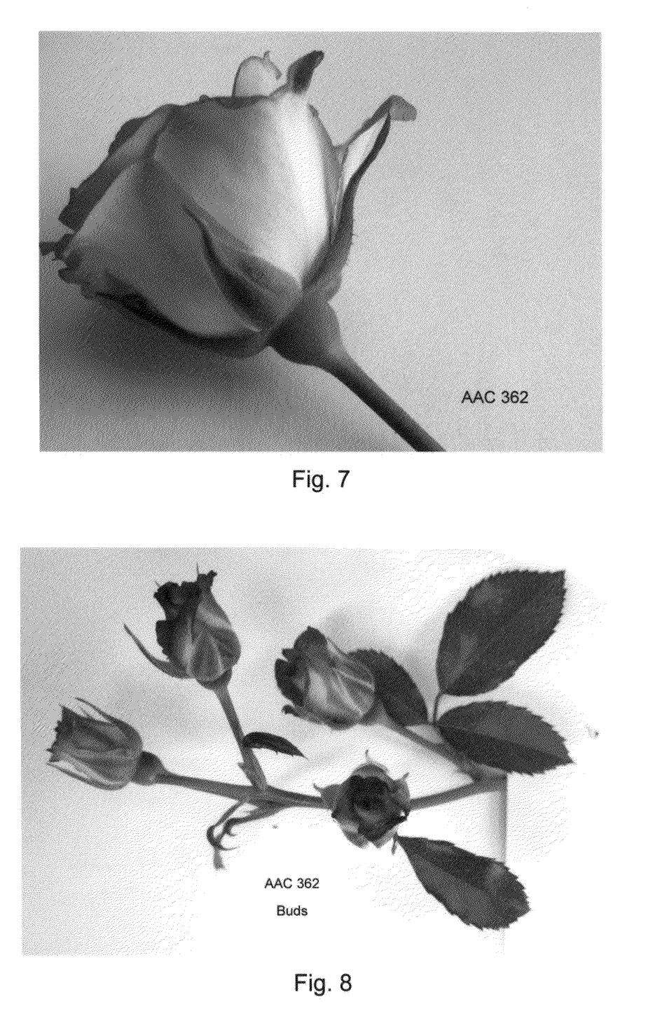

### DETAILED BOTANICAL DESCRIPTION OF THE INVENTION

The variety is uniform and stable. No off-types, variants or mutations have been found to date. Characteristics for ‘AAC 362’ were retained in multiple generations of asexual reproduction through the use of vegetative cuttings.

The RHS Color Chart of The Royal Horticultural Society has been used for color identification. The description is based on observations taken in July on five plants, approximately two years of age, growing on their own roots in containers outdoors at Vineland Research and Innovation Centre, Vineland Station, Ontario, Canada during the year 2015.
The Plant:
   Habit.—Growth type — compact. Growth habit — semi-upright, bushy. Plant height — about 51 cm on average. Plant width at widest point — about 63 cm on average.
   Stem.—Branching — well branched. Texture — smooth young, smooth mature.
   Internodes.—Av. Distance between internodes on mature stems — about 6 mm on average.
   Prickles.—Present or absent — occasionally present. Incidence — average of 0-4 per each 10 cm of stem. Color of immature prickles — near Red Purple Group 59A. Color of mature prickles — near Greyed Yellow Group 161A. Shape — linear to weakly concave. Length of young prickle — average between 4-11 mm. Length of mature prickle — average between 3-12 mm. Color of young prickle — near Red-Purple Group 59A. Color of mature prickle — near Greyed-Yellow Group 161A.
   Foliage.—Arrangement — alternate, compound. Number of leaflets — 3-5. Leaf length mature leaf — about 11.5 cm on average. Leaf width mature leaf at widest point — about 9.2 cm on average. Terminal leaflet length — about 7 cm on average. Terminal leaflet width — about 4 cm on average. Quantity — average 3 leaves per 10 cm of mature stem. Color of upper leaf surface of young leaflet — near Purple Group N77A. Color of lower leaf surface of young leaflet — near Purple Group N77A and occasionally near Yellow Green Group 146B. Color of upper leaf surface mature leaflet — near Green Group N137A and occasionally near Yellow Group 147A. Color of lower leaf surface mature leaflet — near Yellow Green Group 147B. Shape of leaflet — elliptic. Apex of leaflet — acute. Base of leaflet — obtuse. Margin of leaflet — serrate. Texture of young leaflet — smooth on upper and lower surface. Texture of mature leaflet — smooth on upper and lower side. Leaf glossiness young leaflet — glossy. Leaf glossiness mature leaflet — moderate glossy. Venation leaflet — pinnate.
   Stipule.—Length — about 15.7 mm on average. Width — about 13.5 mm on average. Color upper surface of stipule — near Yellow Green Group 146A. Color lower surface of stipule — near Yellow Green Group 146B. Margins — serrate with stipitate glands. Texture — smooth. Shape at apex — acuminate. Shape at base — attentuate.
   Petiole.—Length (stem attachment to first leaflet) — about 32 mm on average. Diameter — about 1.54 mm on average. Color upper side — near Green Group 137A. Color lower side — near Green Group 138A. Surface texture upper side — moderately grooved with some stipitate glands. Surface texture lower side — a few small prickles.
   Rachis.—Length (base of petiole to base of terminal leaflet) — about 76 mm on average. Diameter — about 1.32 mm on average. Upper surface color — near Green Group 137A. Lower surface color — near Green Group 138A. Surface texture — grooved with some stipitate glands and occasionally prickled.
Flower:
   Flower type and flowering habit.—Type — double. Flowering habit — repeat blooming. Flower bearing — solitary and in clusters of 3 flowers. Longevity — long-lasting, more than 2 weeks.
   Flower bud as sepals break.—Shape — ovoid to pointed. Length — about 25.5 mm on average. Width — about 13.5 mm on average. Bud color, upper surface — margin near Red-Purple Group N57A, middle near Yellow Group 5D, base near Yellow Group 5A. Bud color, lower surface — margin near Red-Purple Group N57C, middle near Yellow Group 5A and 5D, base near Yellow Group 4A.
   Flower.—Flower shape — symmetrical rosette flowers. Flower diameter — about 57 mm on average. Flower height — about 22 mm on average. Fragrance — weak.
   Petals.—Shape — obovate. Margins — entire. Apex — round. Base — obtuse. Curvature of longitudinal axis — weakly reflexing. Size — length about 26.5 mm on average, width about 24.8 mm on average. Thickness — firm. Texture — smooth, velvety. Arrangement — imbricate.
   Petal color.—When opening, upper surface — base near White Group 155A, middle near Red Group 53C, margin near Red Group 53A. When opening, lower surface — base near White Group 155A, middle near Red Group 54B, margins near Red Group 53A. Fully opened, upper surface — base near White Group 157C, middle near Red Group 54A with occasional Green-White Group 157C, margins near Red Group 53C. Fully opened, lower surface — base near Green-White Group 157D, middle near White Group 157C with occasional Red Group 55B, margins near Red Group 53D. When fading, margins near Red Group 53D fading to Near Red-Purple Group 60A.
   Petaloids.—absent.
   Peduncle.—Surface — smooth. Hairs — occasional hairs and stipitate glands. Strength — moderately strong. Length — about 32.7 mm on average. Diameter — about 2.3 mm on average. Color — near Yellow-Green Group 144A with anthocyanin coloration near Purple Group 187B.
   Sepals.—Quantity — 5. Shape — lanceolate. Apex — acuminate. Base — truncate. Margins — few extensions with stipitate glands along margins. Size — length about 21 mm on average, width about 7 mm on average. Hairs — fine pubescence on upper and lower surface. Color upper surface — near Yellow Green Group 146B. Color lower surface — near Yellow-Green Group 146C, may have weak anthocyanin coloration near Red-Purple Group 59B.
   Receptacle.—Shape — globular. Surface — smooth. Width — about 7.4 mm on average. Height — about 7.7 mm on average. Color — yellow Green Group 144A with weak anthocyanin coloration near Purple Group 187B.
Reproductive organs:
   Pistils.—Quantity — Average 44. Stigma location — below anthers. Stigma color — near Yellow Group 11 B. Style length — about 5 mm on average. Style color — Upper half near Greyed-Purple 185B, Lower half near Green-White 157C.
   Stamens.—Stamen quantity — average 68 per flower. Anther length — average 2 mm. Anther color — Yellow-Orange Group close to 22A. Pollen — present. Filament color — Yellow Group close to 11A. Filament length — average 4 mm.
   Fruits.—Hips. Quantity — about 1 per flower. Size (mature hip) — not observed. Color (mature hip) — not observed.
   Seeds.—Quantity — about 17 per fruit.
Disease resistance: The variety is resistant to powdery mildew Sphaerotheca pannosa, downy mildew Peronospora sparsa, rust Phragmidium spp., Cercoprora rosicola and black spot Diplocarpon rosae as a potted plant under normal growing conditions. The ‘AAC 362’ variety has not been observed under all possible environmental conditions to date. Accordingly, it is possible that the phenotypic expression may vary somewhat with changes in light intensity and duration, cultural practices, and other environmental conditions.

What is claimed:

1. A new and distinct variety of Rose substantially as herein illustrated and described.
